(19) United States
(12) Patent Application Publication
(10) Pub. No.: US 2008/0282026 A1
(43) Pub. Date: Nov. 13, 2008

(54) Bioprocess data management

(75) Inventors: Mark Selker, Los Altos Hills, CA (US); Barbara Paldus, Woodside, CA (US); Charles Kamas, San Jose, CA (US)

Correspondence Address: Herbert Burkard, BLDG. 1, 3350 Scott Blvd., Santa Clara, CA, 95054, US

(73) Assignee: Finesse Solutions, LLC.

(21) Appl. No.: 12/150,806

(22) Filed: May 1, 2008

Related U.S. Application Data
(60) Provisional application No. 60/928,179, filed on May 8, 2007.

(51) Int. Cl. G06F 12/00

(52) U.S. Cl. 711/103; 711/E12.001

(57) ABSTRACT

A data management system for a biological process, comprising:
a. a single-use component,
b. a tag assembly, including a non-volatile memory storage component, that is associated with the single-use component,
c. the memory storage component including a unique identification and a memory, and at least one data element that describes a key performance or control parameter of the single-use component
d. a memory reader useable to obtain the identification from the memory storage component

### RELATED APPLICATIONS

This application claims priority from co-pending, commonly assigned Provisional Application Ser. No. 60/928,179, filed May 8, 2007.

### BACKGROUND OF THE INVENTION

Over the last several decades, biotechnology has become increasingly fundamental to our society and now has a major impact on the production of food, medicine, fuel, and materials. This importance and influence on our day to day lives has lead to a desire to better monitor and control the processes used to implement this technology. In part due to these reasons, and to end a stagnant period in the technological advancement of drug development, the US FDA has created the PAT (Process Analytical Technology) initiative (http://www.fda.gov/cder/OPS/PAT.htm). This initiative, which will likely become part of the Code of Federal Regulations (CFR), encourages not only large pharmaceutical manufacturers and also smaller modern biotech companies to bring new technological advances into mainstream use to help modernize and optimize biotech manufacturing. Much of the impetus for the PAT initiate is to bring about advances in monitoring and control so that drug manufacturing is safer, more repeatable, more transparent, and less expensive and thereby protect the public. For example, in the “Process Control Tools” section of the PAT guidance document, it states that:

“Strategies should accommodate the attributes of the input materials, the ability and reliability of the process analyzers to measure critical analytes, and the achievement of process endpoints to ensure consistent quality of the output materials and final product.” Design optimization of drug formulation and manufacturing and processes within the PAT framework can include the following steps:
Identify and measure critical material and bio-process attributes relating to product quality
Design of a process measurement system that allows real-time or near real-time (e.g. on-line or at-line) monitoring of critical bio-process/product attributes
Design process controls that enable adjustment to ensure that critical process parameters are controlled
Develop mathematical relationships between product quality attributes and measurements of critical material and process attributes

Much of this can be summarized to mean that by using advanced monitoring of materials used and process variables (e.g.: pH, dissolved oxygen, dissolved CO₂, glucose, glutamine, lactate, ammonia) mathematical models of a bio-process can be created. Through the use of these models, the process yield can be predicted and thereby lead to optimized growth runs even if every parameter is not fully understood. Once monitoring systems are in-place and models created, advanced control systems can be used to implement the optimization procedures.

In the future, for a typical microbial or cell growth run to conform to the PAT strategies as outlined above, it is likely that all the raw materials and also the data used in the growth process will need be recorded and tracked. For instance, the growth media manufacturer's formulation specifics, lot data and manufacture date will need to be logged so that issues like contamination, expiration, or other factors affecting quality or performance can be tracked. The same will be true for the actual cell line used, the pH buffer employed, the glucose feed, the sensor manufacturing data, and other inputs. As the trend towards disposable bioreactors, disposable sensors, and other disposable materials mature and become a major part of the manufacturing chain these items will need to be tracked as well.

### BRIEF DESCRIPTION OF THE DRAWINGS

FIGS. 1a and 1b are flow charts showing a two different process flows for using a radio frequency identification (RFID) tag as a tracking system for single-use bioprocess components: (a) prior art use flow, versus (b) use flow in accordance with two alternative embodiments of the present invention. It should be noted that although the present system will be referred to as a “data management” system it applicability encompasses process and process component monitoring (tracking and/or calibration) and also control of a bioprocess.

FIG. 2 is a schematic showing an example of a single-use bioreactor tracking system in accordance with the present invention.

FIG. 3 shows the block diagram typical gamma radiation resistant ferro-electric random access memory (FRAM) nonvolatile memory chip

FIG. 4 is a schematic showing portions of a data management system in accordance with the present invention which can suitably utilize a FRAM chip.

FIGS. 5a and 5b show two examples of single-use bioreactor tracking systems and their integration into the overall data management and control system: (a) prior art data flow, versus (b) data flow in accordance with the present invention.

FIG. 6 shows a part of a bio-process control system in accordance with the present invention where the disposable element is packaged in a bag to which a RFID tag is attached after sterilization.

FIG. 7 shows a part of a bio-process control system in accordance with an alternative embodiment of the present invention where the RFID is directly attached to the disposable element (e.g., a dissolved oxygen probe) prior to packaging or sterilization, and the tagged disposable element is incorporated within a disposable assembly.

FIG. 8 shows a flow diagram accordance with the present invention showing how to implement label security, to ensure that a single-use component is used only once.

FIG. 9 shows overall and also end and partial cut away side views of a disposable sensor assembly suitable for the practice of the present invention.

### DESCRIPTION OF THE INVENTION

Our invention specifically addresses the need for automated data acquisition by a control system in bio-process manufacturing. For the tracking of any element (e.g., sensor, other component or bio-process ingredient) used in a bio-process, and in order to adhere to the concepts put forth in the PAT initiative, the bioprocess data management (control) system will need to record information that contains, but is not limited to:
1. Calibration or performance data
2. Serial and lot numbers
3. Material certifications
4. Aging information

This information can be automatically loaded into a control system or a transmitter that interfaces with the element to be interrogated using a variety of means which will be discussed below. A transmitter here connotes a device that: i) connects to a probe or non-volatile memory device and supplies it with power, ii) can access the probe or read stored information, and iii) has a human/machine interface (HMI) so that the data can be displayed and understood. After the data is retrieved, it can be utilized by the control system or by the transmitter to optimize the bio-process performance or the data can be displayed and/or logged as part of the data management system. For example, a sensor such as a dissolved oxygen or pH sensor can have its calibration data automatically retrieved in this way. The optimal control algorithm, including growth and feeding strategy, can be automatically implemented if the cell line and growth medium are known, provided only that this information is preprogrammed into the control system). Additionally, any regulatory agency information required can be recorded with the growth run data, provided the material certifications and lot numbers containing this information are automatically read into the system from (?) the non-volatile memory device or other information storage device.

The information required to describe, control, and/or automate a modern biotech process will vary in both scope and quantity. Depending on the volume and sophistication of the data, it can be recorded and read back using a variety of methods. These methods include:
1. RFID chip
2. Nonvolatile memory/EEPROM
3. Internet download
4. Other means to semi-automatically read labels or tags such as holographic stored data markers or fluorescent nano-tags.

The data itself can be embedded in a label, tag, non-volatile memory (e.g.: FRAM), or RFID or surface acoustic wave (SAW) chip.

The prior art (e.g., US2005/0205658 or US2007/0200703), primarily describes a data tracking system, wherein a serial number is encoded in a RFID tag that is attached to equipment or components being monitored. The RFID tag is used to retrieve product information such as the lot number, date of manufacture, materials certificate numbers, and expiration date, from a database on a PC over a secure internet link. The RFID tag can also have read-write capability, so that the tracking system can capture data relating to the exposure of the equipment or component to processes or environments that can damage it, such as sterilization by autoclaving or chemical cleaning. The RFID tag is resistant to these cleaning processes and can be re-read many times during the course of the use of the component or equipment. The overall purpose of the prior art is to track the aging of the equipment or component, so that its failure date can be predicted for scheduled maintenance, and it can automatically be re-ordered and restocked. The prior art describes collecting data from many samples into a database, in order to estimate the useful life and time to replacement for the component or equipment.

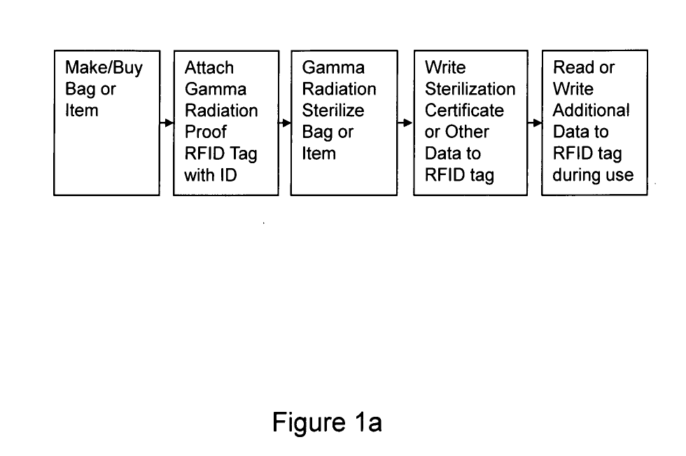

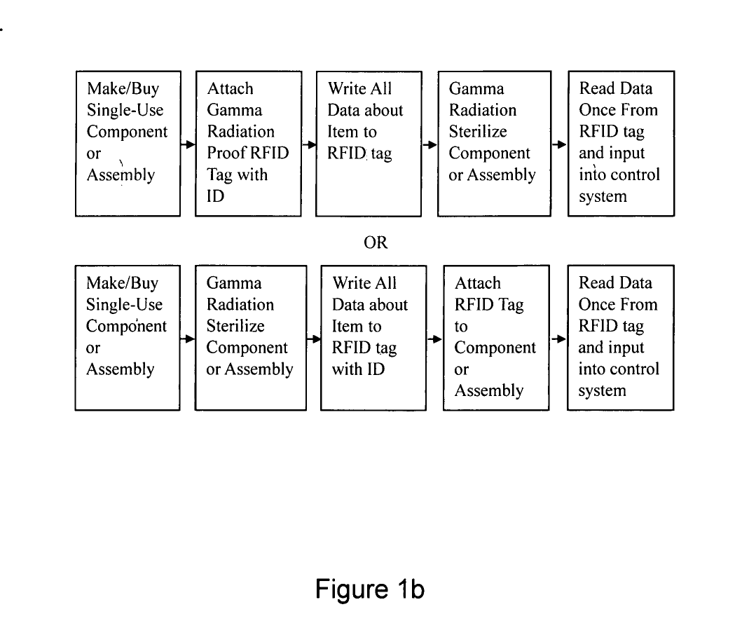

The prior art pertaining to RFID tags used on single-use bio-process equipment or components (e.g., US2008/0024310A1) that are sterilized by gamma irradiation, specifically states that the product tracking information such as serial and lot numbers should be stored on the gamma radiation resistant portion of the tag, but that additional information, such as the radiation dose, is entered on the tag post irradiation. FIG. 1a illustrates the process flow for the RFID tags described in US2008/0024310A1. Therefore, the use case of the tag is identical to the above-indicated patent with the proviso that a portion of the RFID tag memory must be gamma radiation resistant, a requirement that is satisfied by the FRAM technology utilized by companies such as Fujitsu and others. In contrast, the present invention describes labels, including but not limited to RFID tags, where all the information pertaining to the component is entered either prior to or after the final sterilization step, rather than as a sequence during the manufacturing or assembly process for the component (e.g., filling a bag with media, or inserting a sensor into a bioreactor liner bag). Two alternative embodiments of process flows for the present invention is shown in FIG. 1b. In either embodiment the user starts with a single use component (or assembly). In a first embodiment the user: i) attaches a gamma radiation proof tag (label) to the component, ii) enters the appropriate data concerning the component on the tag, iii) gamma radiation sterilizes the component, and iv) reads the data from the tag and inputs the data into the bioreactor control system. In an alternative embodiment, the user: i) first sterilizes the component, ii) enters the appropriate data concerning the component on a tag, iii) attaches the tag containing the data to the already sterilized component, and iv) again reads the data from the tag and inputs the data into the bioreactor control system. The difference is whether the sterilization preferably takes place before or after attachment of the tag which determines by whether the tag needs to be sterilization resistant. In a particularly preferred embodiment the data will contain an encrypted Universal Resource Locator (URL) so that proprietary data can be transferred in paperless fashion.

Unlike the present invention, the prior art does not describe or suggest a label or tag that carries process-specific or sensor calibration data, and is also usable to control a bio-process and/or measure parameters of the bioprocess in real-time. The prior art also assumes that the data is both written to and entered from an external database rather than a transmitter and/or controller directly associated with the bio-process and the component being used. Finally, the prior art assumes that the RFID is writable (can be written to) and that the user will input more than one process event on the tag. In the present invention, the labels and tags are exclusively associated with single-use components, and are therefore read only once, at the start of the bio-process, because they are discarded after the bio-process is complete. Other prior art pertaining to water quality monitoring tools (e.g., U.S. Pat. No. 7,007,541) is primarily aimed at re-usable sensors whose calibration constants change with aging or interchangeable sensors where the re-usable sensor heads are each unique enough that their parameters need to be accounted for systematically.

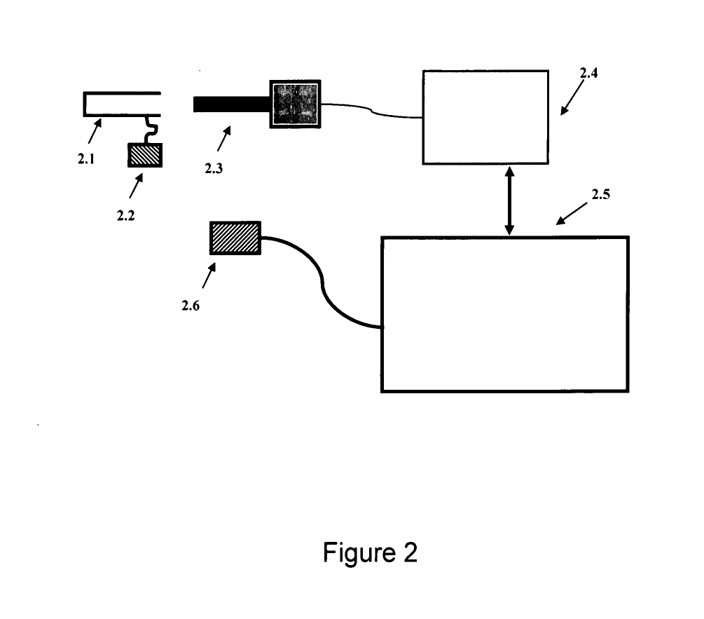

When using a semi-automatically-readable (take to reader) label (tag) such as a set of magnetic stripes (or equivalent marking system) or a memory device based on SAW (surface acoustic wave) chips the reading of the data will advantageously be semi-automated. In the present context semi-automated means that the user will not need to manually enter the data describing the component, and the user will only need to bring a reader into sufficiently close proximity and with a specific orientation in order to accomplish the data transfer to the reader. An example in accordance with the present invention is shown in FIG. 2. In FIG. 2, 2.1 is a disposable element on which an encoded label 2.2 resides, 2.3 is a re-useable element, and 2.4 is the transmitter to which 2.3 is connected, 2.5 is an automation system that consists of both control software and hardware. A label reader 2.6 is shown connected to the automation system. Since the system is in communication with the transmitter 2.4, the label 2.2 information can be used by the transmitter. The disposable element 2.1 can, for example, be a disposable sensor, a disposable (single use) bioreactor vessel, a container of a particular microbe or cells from a cell bank, growth medium, pH buffer, or any other input or process variable used in a growth run or similar biotechnology process.

Another level of automation is the use of non-volatile memory storage component such as FRAM (ferro-electric based random access memory) or an EEPROM (Electrically Erasable Programmable Read-Only Memory) chip (equivalent functionality to a label) to store data and provide an interlock for the system. A system using non-volatile memory chips such as FRAM or an EEPROM can be employed for any component that is plugged into (i.e., is physically connected to) the system. For instance, if using a disposable bioreactor vessel and/or a set of disposable sensors, the disposable elements can be plugged into the data management (control) system of the present invention. For example, if the bioreactor under study is a disposable bioreactor or bioreactor using disposable elements, the recorded information regarding the date of manufacture, the materials used and their certifications, (e.g.: growth media, sensor calibration data etc.) can all be automatically loaded into the control system memory from the nonvolatile memory after it is plugged into the system. A FRAM based nonvolatile memory, for example, is inexpensive and therefore can be readily disposed of with the disposable component after a single use.

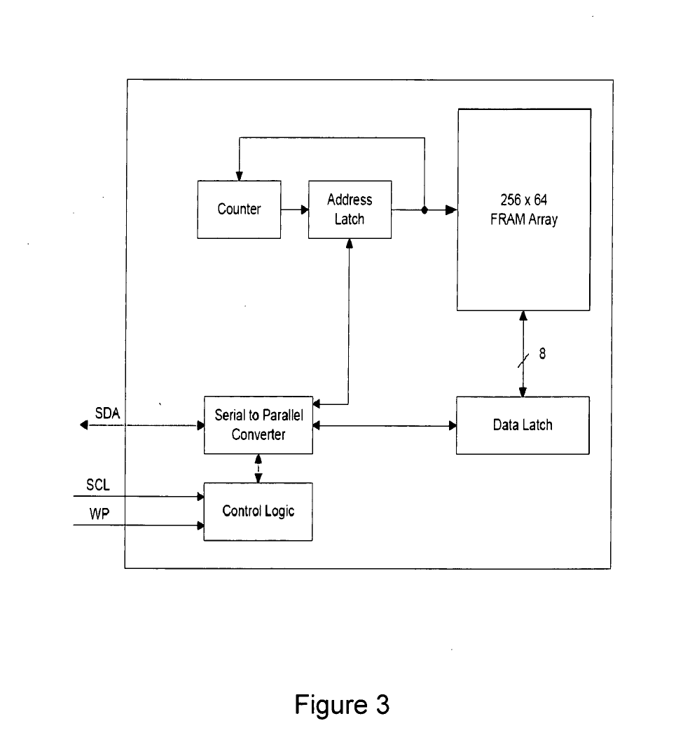

The gamma radiation resistant, nonvolatile memory allows for the transfer of calibration or other information from the factory to the apparatus without concern for the possibility of operator error. This is a significant advance over the current state of the art which calls for an operator to enter this type of information via a keypad or by scrolling through alpha numeric characters one at a time. Any particular (or all) information can be encrypted in order to verify its authenticity and to protect it from tampering. This also allows the manufacturer to provide a unique identification code for each device/component for traceability purposes. This unique identification code thus allows the data management (control) system to control the number of times, duration, or conditions under which the component is used, and can therefore be used to prevent misuse and fraud. Such misuse can, for example, include trying to use pre-sterilized disposables more than once. FIG. 3 shows the first page of the data sheet of an FRAM-based, non-volatile memory chip. EEPROM's can also be obtained that are gamma radiation resistant, but to date these devices are more expensive and therefore somewhat less appealing in certain cases.

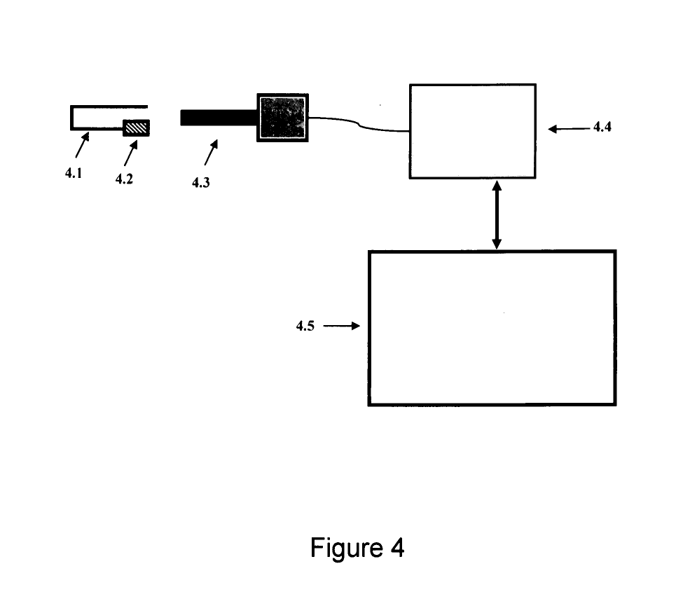

FIG. 4 depicts a typical application using a control system in accordance with the present invention. In FIG. 4, 4.1 is the disposable element, 4.2 is the FRAM or equivalent non-volatile storage element, 4.3 is a re-usable element or reader into which 4.1 is connected, 4.4 is a transmitter which can optionally interact with either the re-usable element 4.3 or with the FRAM. When the disposable element 4.1 is connected to the reusable element 4.3, the data in the FRAM is read and processed as discussed above. The automation system, 4.5, is connected to the transmitter, and can act as the master controller or the repository for data read into the transmitter. Element 4.1 can be a disposable sensor, a disposable element for a bioreactor such as a valve or bag or a similar single-use item. As many of the disposable or single-use components in a bioprocess are relatively small, the size of the FRAM can be important. Many non-volatile memory storage components (chips) are physically large in order to help enhance their gamma radiation resistance which can pose a problem for locating the memory device on the disposable component. In general, chips that are similar in shape to a standard SOIC (small outline integrated circuit) package or a flat-pack with leads coming from all 4 sides of the chip will advantageously be utilized. The optimal chip will therefore preferably have a surface area no larger than 1 cm₂ and be no thicker than 1 mm and most preferably be approximately 6 mm×6 mm and 0.5 mm thick.

A similar result can be accomplished through the use of an RFID-based tagging system. Similar to the nonvolatile memory and the label systems described above, this embodiment of the present invention enables one to perform the following functions:

1. Transfer data and information from the manufacturer's calibration database or data storage to the control system without operator error.

2. Eliminate time consuming manual data entry via a keypad or by sequentially scrolling through alpha numeric characters one at a time.

3. Encrypt data and information to guarantee its authenticity.

4. Transfer information without out any physical contact or particular orientation of the RFID tag.

5. Provide a log of each unique identification tag for traceability, as well as to minimize possibility of misuse or fraud.

A benefit resulting from using an RFID tag system is that the identification system does not need to be physically attached to the disposable element. This method enables one to tag the disposable element or disposable sensor instrument (or the package that contains it), such that it can be tracked from manufacturing to final use. The RFID tag preferably includes a unique identification number. The tag also carries the aforementioned information in its nonvolatile memory. The information is advantageously encrypted and check-summed in order to prevent tampering and/or invalid calibration. In one example (FIG. 1b, top process flow), the RFID tag is attached to the disposable element and product specific information is entered on the tag prior to sterilization. The RFID tag is then sterilized together with the disposable element (component). Alternatively, the RFID tag can be applied to the component or its packaging or traveler/paperwork after it has been sterilized, and when all necessary information about it is known (FIG. 1b, bottom process flow). This is particularly useful if a sterile or bio-inert requirement exists. Tags that can survive gamma radiation are often larger and more expensive, so it can sometimes be preferable to apply the tag to the package or to a traveler (a record of manufacturing processes and component serial numbers) and avoid opening and contaminating the disposable element. The disposable element can then be sterilized in place with radiation, while the traveler with the RFID tag is then be brought into proximity to the reader and the data entered automatically. For example, if this is a sensor, it will be the calibration data and other applicable manufacturing information; for a disposable bioreactor, it can be the films used; for growth medium, it can be the lot and serial number for process tracking. This RFID tag system can be used with any disposable bio-process components that will benefit from having information managed. The size of the RFID tag can be important as the efficacy is related to the size. The larger the RFID tag's area, typically the larger the antenna of the tag and hence the greater distance it can be from a reader and still be read. However, smaller tags with the antenna constructed of multiple loops are also effective and therefore preferred. In general, the tag needs to be large enough to satisfy the distance requirements for its use, yet small enough that it can still be packaged with the single-use component which needs to be tracked, calibrated, or otherwise have its data managed. The RFID tag will therefore preferably have a substantially planar configuration and a surface area no greater than about 150 cm²

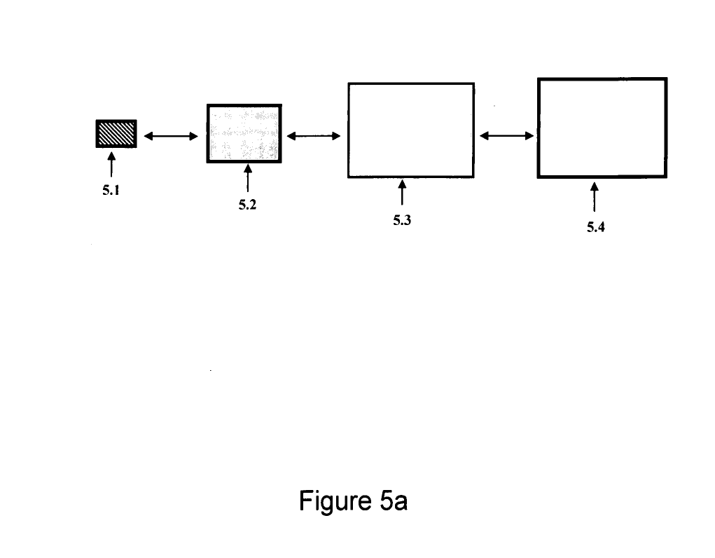

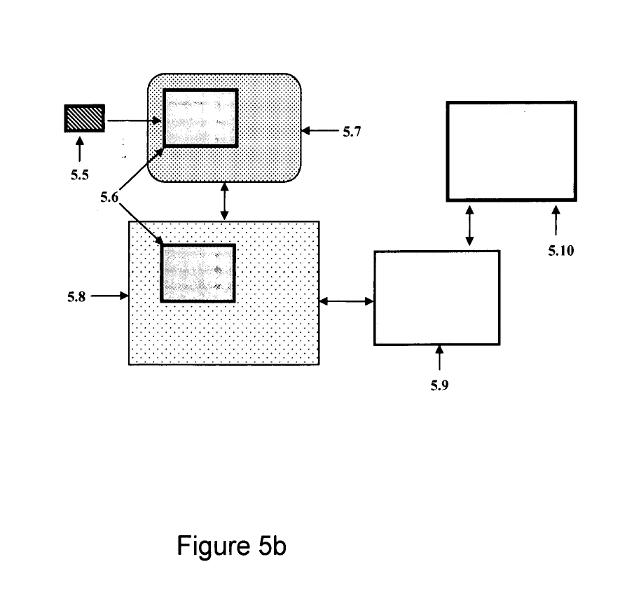

In the prior art techniques, the data flow to and from the label on the disposable element bypasses the automation system associated with the bioprocess in which the disposable element is used. FIG. 5a illustrates the data flow as described in published applications US2005/0205658, US2007/0200703, and US2008/0024310A1. In these cases data from RFID tag 5.1 is read or written by reader 5.2 to computer 5.3 that links into an external database 5.4. Database 5.4 is either stored on computer 5.3 or is external, with Ethernet access from computer 5.3. Such data flow is appropriate for a system that is associated with manufacturing quality, materials requirements planning, or enterprise resource planning systems. Such a prior art system can generate a database that provides information to estimate useful service life and time to failure for components, as well as an ability to re-order inventory. However, such a database is only useful for the control of a bio-process system in the event of a process failure, when materials certificates and serial numbers must be accessed for a root cause analysis of the failure.

In the present invention, as in the embodiment shown in FIG. 5b, the data flow from the disposable element label 5.5 occurs through reader 5.6 into either transmitter 5.7, whose output is connected to controller 5.8, or directly into controller 5.8. The process data containing the label information is then saved in 5.9 (the system historian or historical database) as part of the batch record, or as a process parameter. The data from label 5.5 is used either by transmitter 5.7 or controller 5.8 during the bio-process, in order to affect control of the bio-process. For example, calibration constants can be used by the transmitter to calculate sensor output values that are sent to the bio-process automation system, which then actuates pumps or mass flow control valves; or the amino acid concentration in the media of a pre-filled bioreactor bag is used by the control system to predict feeding and cell growth rates after inoculation. In both of these examples, the data from the label/non-volatile memory is actively used to control the bio-process, and generates additional, associated process data that can be used to characterize the effectiveness of the disposable element in the process for future runs. This use of disposable labels is equally applicable to upstream (cell culture/fermentation), downstream (purification), or fill-finish bio-processes.

Furthermore, the control system 5.8 can be linked to a materials requirements planning system within the fabrication facility 5.10, such as SAP or Oracle, update the inventory levels automatically after the completion of the process using the disposable element, and input process feedback into the plant management system. Unlike the prior art, which requires human intervention to an external database, this inventory management can be performed completely automatically using the data management system of the present invention.

In the present invention, the ID number that is stored on the label or other non-volatile memory may correspond to product specification information for the component, such as materials certifications, lot numbers, manufacturing date, and/or sterilization records. This information can be stored in a remote database, for example, a section of the supplier's database that is only accessible by the end user or OEM customer. In contrast to the prior art, where the informational database must be accessed manually by the user, in the present invention, the database URL address and an optional encrypted key-code for remote database access are also stored on the label or tag and are read out by the transmitter or automation system. If either transmitter or automation system is connected to the internet via the Ethernet, it can automatically access the URL, enter the optional key-code, and automatically gain access to the database information, in order to download it and store it in the process batch record. Alternatively, if the bio-process automation system and/or transmitter are programmed to have their own user ID and password to the database and the URL has been already entered into their memory, only the component's ID number is required from each label or tag, and database access remains automatic.

Most systems in accordance with the present invention will utilize a disposable element such as a sensor element or a disposable element that comprises a sensor element, a reusable component that holds the electronics measuring the sensor response and which interfaces to the transmitter, and also an RFID tag having both a unique identifier and a nonvolatile memory element. A process for utilizing the system of the present invention would proceed according to the following steps:
1. The disposable (e.g.: sensor) element is first calibrated using a known method.
2. After the calibration and performance data for the disposable element is generated, it needs to be associated with the single use component for which the data was generated in the bio-process.
3. The disposable element is sealed in a bag with a visible identifying number or tag, such as a paper label.
4. The bag containing the disposable element is gamma irradiated and a RFID tag is applied to the outside of the bag.
5. A computer program encodes the calibration information on the RFID tag, along with any additional information pertaining to the disposable element, such as material certificate numbers, batch numbers, etc.
6. This information is stored in the RFID tag's nonvolatile memory elements.
7. The RFID tag's unique identifier is recorded visibly on its exterior for ease of identification.

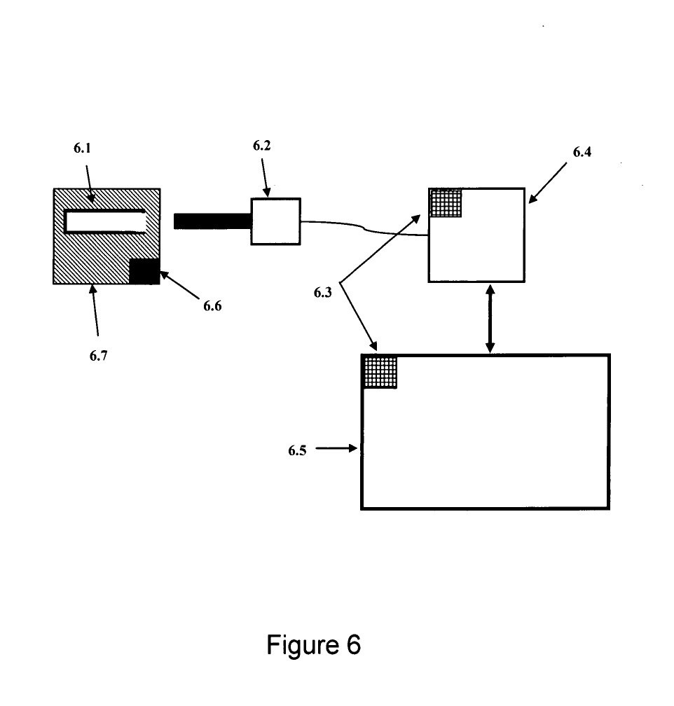

Once the disposable element is ready to be used, it is taken to the reusable element where a scanner (reader) reads the data from RFID tag, both the unique identifier and also the nonvolatile memory elements. The reusable element will have an associated transmitter or processor that decodes and applies the information it has read from the RFID tag. The disposable element can now be used with minimal intervention by the end user. If this is a sensor, it is now ready to take measurements; if it is disposable bioreactor system then all of the relevant data on the bag, the growth media, configuration, batch ID, etc., is now entered into the control system. This is shown in FIG. 6 where 6.1 is the disposable element, 6.2 is the reusable element, and 6.3 are RFID readers which can be located either in the transmitter 6.4 or the automation system 6.5. The RFID tag 6.6 is affixed to a preferably bio-inert or sterile container 6.7 for the disposable element. This tag can also be affixed to a manufacturing traveler or equivalent paperwork that is brought near to the proximity reader.

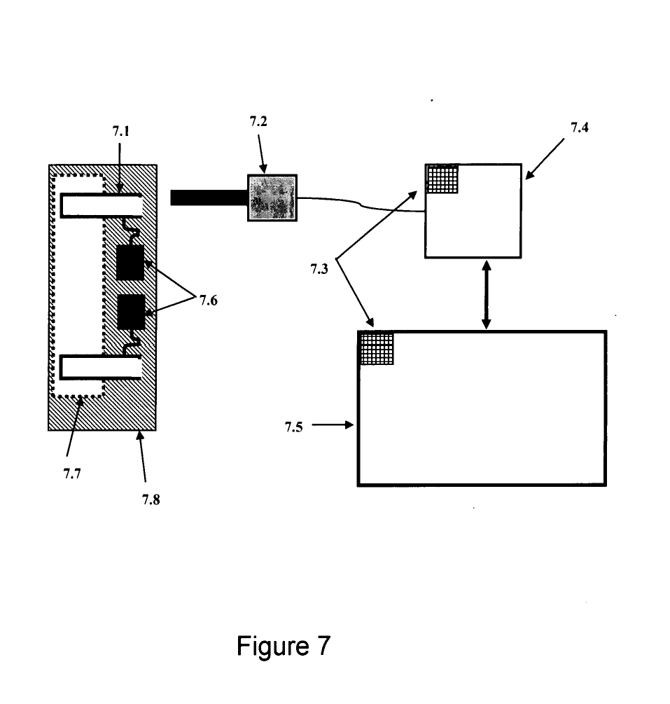

Note that in an alternative embodiment illustrated in FIG. 7, 7.1 is the disposable element, 7.2 is the reusable element, and 7.3 are the RFID readers which can be located either in the transmitter 7.4 or the automation system 7.5. The RFID tag 7.6 is directly attached to the disposable element 7.1, and the calibration or other data is written onto the non-volatile memory of the RFID tag using a computer. The disposable element may then either be integrated into a larger assembly 7.7, such as a bioreactor bag for a disposable sensor or component and packaged in a bag 7.8, or be separately and directly packaged in a bag 7.8. The assembly 7.7, including any attached RFID tags, is then sterilized, either individually, or as a group on a pallet. When the assembly 7.7 is used in a bio-process, the bag is removed, and each tag is removed from its associated component and scanned into the system.

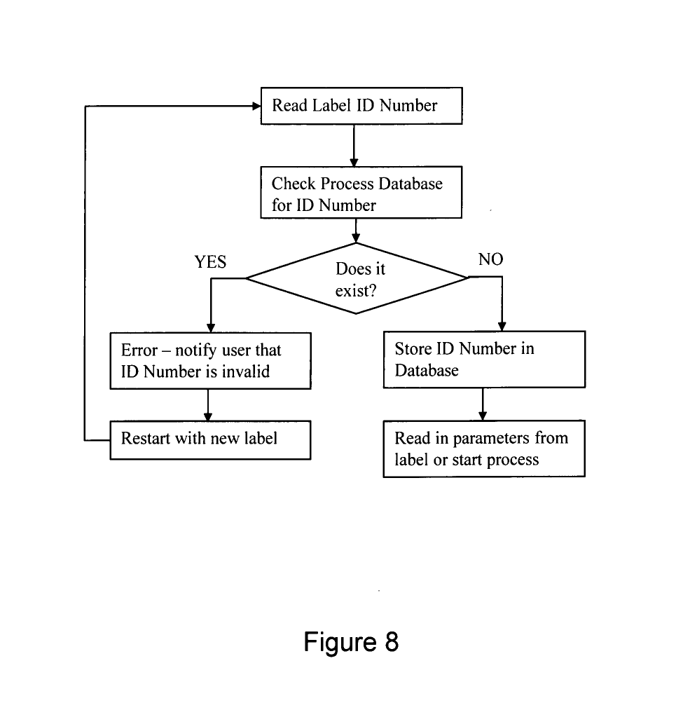

The re-usable element, or the system to which the re-usable element is connected, will also preferably have its own nonvolatile storage. This memory can be used to log the usage of the disposable elements. For example, this usage log can be utilized to verify that the disposable element has never been used before. If the unique identification number has been used before or does not conform to a validation algorithm, the identification is invalidated and a warning to this effect is given through the interfaces. The architects of the system can decide how much or how little to minimize the user's activity. FIG. 8 shows an example of a flow diagram associated with RFID security, so that a single-use component cannot be re-used, and thereby not cross-contaminate a subsequent process.

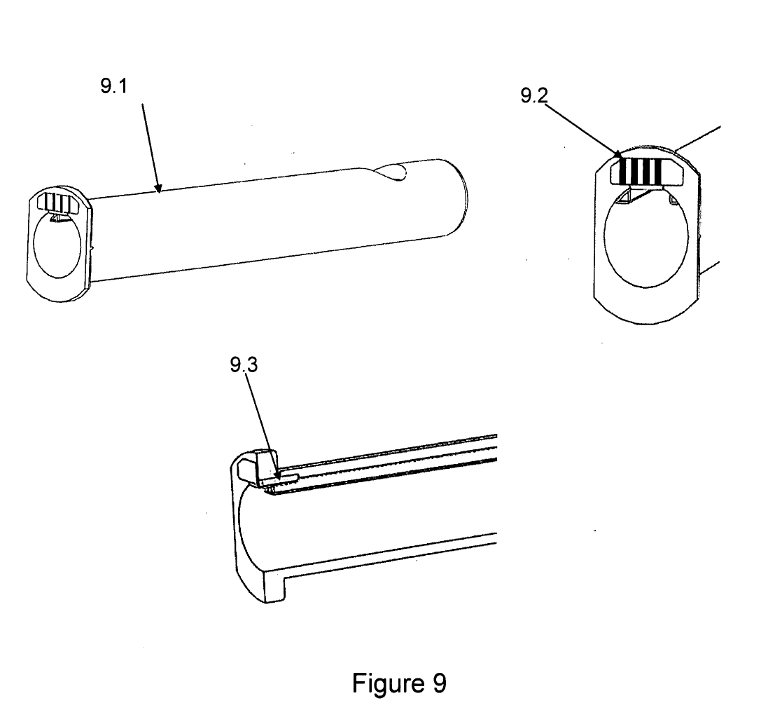

Referring now to FIG. 9, there is illustrated overall (9.1) and also end and partial cut away side views of a disposable sensor assembly suitable for the practice of the present invention. 9.2 denotes electrodes which enable the non-volatile memory (such as a FRAM) 9.3 to interface with the transmitter such as that designated as 5.7 in FIG. 5.

1. A data management system for a biological process, comprising:
a. a single-use component,
b. a tag assembly, including a non-volatile memory storage component, that is associated with the single-use component,
c. the memory storage component including a unique identification and a memory, and at least one data element that describes a key performance or control parameter of the single-use component
d. a memory reader useable to obtain the identification from the memory storage component

2. The data management system of claim 1, wherein the single-use component is an assembly of a plurality of single-use components, each of said components having its own tag.

3. The data management system of claim 1 wherein the memory reader is a transmitter.

4. The data management system of claim 3 wherein the transmitter is used for the measurement of a bio-process relevant parameter

5. The system of claim 4 wherein said bio-process parameter is pH, dissolved oxygen, dissolved C0₂, temperature, pressure, level, foam, cell density, cell viability, metabolites such as glucose, lactate, glutamine, glutamate or ammonia, anti-form, additives, amino acids, or a bio-process end product comprising a protein, antibody or plasmid.

6. The data management system of claim 1 wherein the memory reader is a component of the process control system.

7. The data management system of claim 1 wherein the memory storage device is a non-volatile memory that is not adversely affected by gamma radiation.

8. The data management system of claim 7 wherein the memory storage device utilizes FRAM.

9. The data management system of claim 1 wherein the memory storage device is a RFID tag comprising a material not adversely affected by gamma radiation.

10. The RFID tag of claim 9 having a substantially planar configuration and a surface area no greater than about 150 cm²

11. The data management system of claim 9 wherein the memory storage device utilizes FRAM.

12. The data management system of claim 8 wherein the memory storage device is utilizes an EEPROM

13. The data management system of claim 1, wherein the data element is at least one calibration constant for a sensor.

14. The data management system of claim 1, wherein the data element is at least one critical additive for a bio-process media.

15. The data management system of claim 1, wherein additional information is stored in the memory, including at least one of a manufacturing date, a batch number, a lot number, material specifications, material lot number, certifications for sterility, certificates of compliance, size specification, functional specifications, description of device, expiration date, process data, calibration data, lifetime data, composition data, or customer application data associated with the identification number.

16. The data management system of claim 1 wherein the non-volatile memory storage component has a surface area no larger than 1 cm² and is no thicker than 1 mm.

17. A method for data management in a biological process, comprising the steps of i) attaching a gamma radiation proof tag to a single use component applicable to said biological process, ii) entering the appropriate data concerning the component on the tag, iii) gamma radiation sterilizing the component, and iv) reading the data from the tag and inputing said data into the control system for said biological process.

18. A process in accordance with claim 17 wherein at least a portion of said data contains an encrypted URL.

19. A method for data management in a biological process, comprising the steps of: i) sterilizing a single use component applicable to said biological process, ii) entering the appropriate data concerning the component on a tag, iii) attaching the tag containing the data to said already sterilized component, and iv) reading the data from the tag and inputing the data into the control system for said biological process.

20. A process in accordance with claim 19 wherein at least a portion of said data contains an encrypted URL.
